(19) United States
(12) Patent Application Publication
(10) Pub. No.: US 2012/0010469 A1
(43) Pub. Date: Jan. 12, 2012

(54) CATHETER GUIDING TOOL

(75) Inventor: Jean Michel Boyer, Nesles la Montagne (FR)

(73) Assignee: SOMEDICS RD, Issy-les-Moulineaux Cedex (FR)

(21) Appl. No.: 13/151,727

(22) Filed: Jun. 2, 2011

(30) Foreign Application Priority Data
Jun. 3, 2010 (FR) 1054373

(51) Int. Cl. A61B 19/00

(52) U.S. Cl. 600/129

(57) ABSTRACT

A catheter guiding tool includes a handle of the guiding tool, the handle including an inner cavity extending between a proximal end and a distal end of the handle; a flexible wire guide; a cover closing the proximal end of the handle, wherein the proximal end of the wire guide forms a coil spring, the coil spring being positioned in the inner cavity of the handle and held in rotation by the cover, the cover being solidary in rotation of the handle.

The invention relates to a guiding tool, also designed by the term stylet, for a catheter. More particularly, the invention relates to a guiding and positioning tool for a pacemaker probe. In particular, such a guiding tool enables a pacemaker probe to be positioned in a precise location of the heart.

When a pacemaker and one or two probes are implanted, after local anesthesia, an incision is made:
in the deltopectoral groove if the cephalic vein is approached, or
in the direction of the median part of the clavicle, which enables the cephalic vein or the subclavian puncture to be approached.

The distal end of the probe is introduced in the chosen vein until it is positioned in the desired heart cavity (atrium or ventricle).

A permanent pacemaker electrode probe is usually constituted of:
a coiled conductor insulated from the external environment by a sheath made of silicone or polyurethane; or
two coiled conductors sometimes insulated independently from each other. These conductors are insulated from the external environment by a sheath made of silicone or polyurethane.

As the technology used for the construction of the probe is coaxial or collinear, the inside of the conductors presents the appearance of a tunnel by which the guiding tool called a stylet is introduced. The guiding tool comprises a metal wire with a constant diameter generally of between 0.35 to 0.40 mm. The proximal end of the guiding tool is equipped with an operating handle, also called a “retainer.”

When a cardiac probe is positioned, the wire guide is introduced by sliding into the inner tunnel with tight bends of the probe (coiled conductor). The distal end of the wire guide sometimes comes up against the bifurcations formed by the probe. This is particularly due to the metallic material of the wire guide that gives it limited flexibility.

Consequently, when the distal end of the wire guide comes up against an obstacle formed by the probe and as its flexibility does not allow it to pass despite the push force exerted by the practitioner, it may happen that the distal end of the wire guide first pierces the conductive winding by damaging its coils and then, secondly, perforates the insulating sheath to then come into contact with a vein or biological tissue.

In addition, with a conventional guiding tool, the practitioner slides the latter into the pacemaker probe until the distal end of the guiding tool is in contact with the distal end of the probe. When the two ends are in contact with each other, a given distance, substantially equal to 20 mm, is present between the entry of the probe and the distal end of the handle. It often happens that the practitioner increases his push force until the handle of the guiding tool comes into contact with the entry of the probe. The probe then undergoes a critical elongation.

In this context, the invention aims to propose a catheter guiding tool allowing the aforementioned problems from the prior art to be resolved by ensuring, in particular, the guiding and positioning of a catheter in a secured manner.

For this purpose, the object of the invention relates to a catheter guiding tool comprising:
a handle of said tool, said handle comprising an inner cavity extending between a proximal end and a distal end of said handle;
a flexible wire guide;
a cover closing said proximal end of said handle;

Said guiding tool being characterized in that the proximal end of said wire guide forms a coil spring, said coil spring being positioned in said inner cavity of said handle and held in rotation by said cover, said cover being solidary in rotation of said handle.

In other words, the cover enables the handle to be solidary in rotation of the coil spring. Thus, when the handle is subjected to a rotation movement, the coil spring and thus the wire guide is also subjected to a rotation movement.

For the rest of the description, catheter or probe is understood to refer to an instrument in the form of a flexible hollow rod that may be introduced in a natural conduit or blood vessel.

Conventionally, for the entire description:
proximal end of an element is understood to refer to: the end closest to the end of the wire guide intended to be inserted in a catheter;
distal end of the same element is understood to refer to: the end farthest from the end of the wire guide intended to be inserted in a catheter.

When the wire guide is passed through a catheter and as it comes up against a bend, the practitioner will increase the push force so that the wire guide passes this obstacle. Thanks to the invention, the spring formed by the proximal end of the wire guide compensates for the push force so as to not damage the conducting sheath, the insulating sheath and the biological tissues. Such compensation is possible thanks to the presence of the coil spring. A coil spring is used since this type of particular spring obtains significant strokes, absorbs the forces to which it is subjected and returns to its initial position when it is not subjected to a force.

As the spring is held in rotation with relation to the handle by means of the cover, the handle may be used to cause the wire guide to pivot in the catheter so as to more easily pass the bifurcations that the distal end of the wire guide may encounter.

In summary, when the wire guide is inserted in a catheter, the coil spring and the holding in rotation of the wire guide with relation to the handle give the guiding tool according to the invention a great ease of use, enabling the bifurcations imposed by the venous network, formed by the catheter, to be passed.

Advantageously, the wire guide material, the coil spring stroke and the number of coils in the spring are chosen according to the resistance to elongation of the catheter.

Preferably, the coil spring formed by the wire guide presents a stroke of at least 20 mm.

In addition to the principal characteristics that have just been mentioned in the previous paragraph, the catheter guiding tool according to the invention may present one or more of the additional characteristics below, considered individually or according to all technically feasible combinations:
said cover comprises:
   a first substantially cylindrical part able to cooperate with said proximal end of said handle;
   a second part forming a substantially parallelepiped plate situated in the extension of said first part such that the proximal end of said coil spring may be positioned between the peripheral surface of said inner cavity and the external surface of said second part;
said wire guide comprises a groove at the region of its distal end, this groove increasing the flexibility of the distal end of the wire guide such that the latter may easily follow the bifurcations of the catheter;
said wire guide forming the bottom of said groove comprises a diameter substantially equal to 0.25 mm;
said groove comprises a length of between 30 mm and 35 mm;
said distal end of said wire guide comprises a domed head, the peripheral surface of the domed head enables the distal end of the wire guide to be guided during its insertion in the catheter;
said domed head has a length on the order of 4 mm.

Other characteristics and advantages of the invention will clearly emerge from the description given below, for indicative and in no way limiting purposes, with reference to the attached figures, among which:

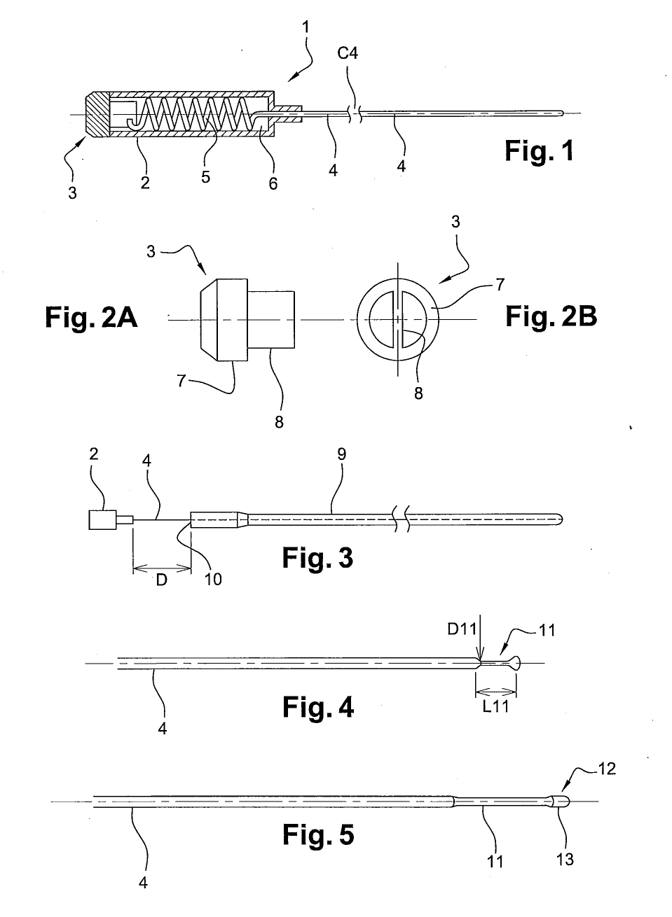

FIG. 1 represents a catheter guiding tool in conformance with the invention;

FIGS. 2A and 2B illustrate a cover utilized in the catheter guiding tool as represented in FIG. 1;

FIG. 3 illustrates a guiding tool as represented in FIG. 1 and implemented within a cardiac probe.

FIG. 4 represents a variation of a guiding tool in conformance with the invention;

FIG. 5 represents another variation of a guiding tool in conformance with the invention.

For reasons of clarity, only the elements essential for understanding the invention have been represented, without respecting the scale and schematic manner. In addition, identical elements found on different figures bear the same references.

FIG. 1 represents a catheter guiding tool 1 in conformance with the invention. Guiding tool 1 comprises, in particular:
a handle 2 (also called support handle) of guiding tool 1;
a cover 3;
a flexible wire guide 4. For clarity of the drawing, in order to be able to represent the distal and proximal parts of wire guide 4, it is represented with a cut C4.

The proximal end of wire guide 4 is wound so as to form a coil spring 5.

Handle 2 comprises an inner cavity 6 extending between its proximal end and its distal end and is suitable for receiving the coil spring 5.

Inner cavity 6 is open at the region of the proximal end of handle 2.

So as to contain the coil spring 5 inside inner cavity 6, the latter is closed by cover 3.

Besides containing the coil spring 5 inside inner cavity 6, the cover also enables handle 2 to be solidary in rotation with coil spring 5.

To do this, as illustrated in FIG. 2, the cover 3 may comprise:
a first substantially cylindrical part 7 able to cooperate with the proximal end of said handle 2;
a second part 8 forming a substantially parallelepiped perpendicular plate in contact with the distal end of first part 7 such that the proximal end of coil spring 5 may be inserted between the peripheral surface of inner cavity 6 and the external surface of second part 8.

This particular layout enables coil spring 5 to be held in rotation with relation to handle 2. As may be seen in FIG. 1, the proximal end of coil spring 5 is slipped underneath the second part 8 forming a plate.

Advantageously, when the wire guide 4 is inserted within a cardiac probe (not represented), the user may, because the wire guide 4 is held in rotation with relation to the handle 2, cause wire guide 4 to pivot by rotating handle 2. This wire guide 4 rotation enables the practitioner to pass some bifurcations formed by the cardiac probe which the distal end of wire guide 4 may come up against.

In addition, by way of a non-limiting example, so as to restrict the coil spring 5 within the inner cavity 6, the first part 7 of cover 3 may be screwed into handle 2. In a different embodiment, the first part 7 of cover 3 may be glued to handle 2.

As previously indicated, the guiding tool 1 according to the invention is suitable for being inserted into a catheter and more particularly into a cardiac probe for positioning the latter in a desired area of the heart. A cardiac probe is generally passed through the venous network presenting bifurcations.

When the distal end of the probe comes into contact with a bifurcation, the practitioner exerts a stronger push force on handle 2 of guiding tool 1 such that the distal end of the probe may pass by this venous bifurcation. Thanks to the invention, there is no risk of damage to the probe or even to the vein since the coil spring 5 aims to compensate for such excess thrust.

The positioning of a probe 9 for a pacemaker is represented in further detail in FIG. 3.

Probe 9 is hollow so as to be able to receive the wire guide 4. Wire guide 4 is then slipped into probe 9 until the distal end of wire guide 4 is in contact with the distal end of probe 9. When the two ends are in contact with each other, a given distance D, substantially equal to 20 mm, is present between the entry 10 of probe 9 and the distal end of handle 2.

If the practitioner increases the push force, the coil spring 5 will compensate for the excess thrust such that probe 9 does not elongate. In other words, the coil spring 5 undergoes deformation by being compressed, and not probe 9.

An advantageous embodiment of the distal end of guide wire that comprises a guiding tool 1 according to the invention is represented in FIG. 4.

More precisely, in such an embodiment, wire guide 4 is equipped with a groove 11 at its distal end.

This groove 11 gives the distal end of wire guide 4 significant flexibility allowing it, during insertion, to follow the possible bifurcations of the catheter within which it is inserted.

By way of a non-limiting example, the diameter of wire guide 4 according to the invention presents a diameter of 0.35 mm and the diameter D11 forming the bottom of groove 11 is substantially equal to 0.25 mm.

In addition, the length L11 of groove 11 is preferably between 30 mm and 35 mm.

Groove 11 may be made by means of a turning operation or by a grinding operation consisting of removing the wire guide 4 material to obtain a given diameter D11 and length L11.

FIG. 5 illustrates another variation of a wire guide 4 that comprises a guiding tool 1 in conformance with the invention.

To increase the ease of passage of guiding tool 1 in a catheter, wire guide 4 is equipped at its distal end with a domed head 12.

The peripheral surface 13 of domed head 12 enables the latter to move easily within a catheter.

To do this, head 12 preferentially presents a length on the order of 4 mm. Such a head 12 length enables it to avoid passing through the coils of the pacemaker probe.

The catheter guiding tool according to the invention was more particularly described in the case of a utilization for positioning a pacemaker probe. However, it is understood that this guiding tool may be used for any type of catheter.

The invention is described in the foregoing by way of example and it is understood that the person skilled in the art is capable of making different variations, particularly in that relating to dimensions of the coil spring, groove or domed head that comprises the wire guide or else the shape of the cover, without necessarily departing from the scope of the invention.

1. A catheter guiding tool comprising:
a handle of said guiding tool, said handle comprising an inner cavity extending between a proximal end and a distal end of said handle;
a flexible wire guide;
a cover closing said proximal end of said handle;
wherein a proximal end of said wire guide forms a coil spring, said coil spring being positioned in said inner cavity of said handle and held in rotation by said cover, said cover being solidary in rotation of said handle.

2. The guiding tool according to claim 1, wherein said coil spring has a stroke of at least 20 mm.

3. The guiding tool according to claim 1, wherein said cover comprises:
a first substantially cylindrical part able to cooperate with said proximal end of said handle;
a second part forming a substantially parallelepiped plate situated in the extension of said first part such that the proximal end of said coil spring may be positioned between the peripheral surface of said inner cavity and the external surface of said second part.

4. The guiding tool according to claim 1, wherein said wire guide comprises a groove at a region of its distal end.

5. The guiding tool according to claim 4, wherein said wire guide forming the bottom of said groove comprises a diameter that is substantially equal to 0.25 mm.

6. The guiding tool according to claim 4, wherein said groove comprises a length of between 30 mm and 35 mm.

7. The guiding tool according to claim 1, wherein said distal end of said wire guide comprises a domed head.

8. The guiding tool according to claim 7, wherein said domed head has a length of about 4 mm.
